(19) United States
(12) Patent Application Publication
(10) Pub. No.: US 2024/0255529 A1
(43) Pub. Date: Aug. 1, 2024

(54) NOVEL ASSAY AND NOVEL METHODS OF TREATING AGING-RELATED CONDITIONS

(71) Applicant: The Progeria Research Foundation, Peabody, MA (US)

(72) Inventor: Leslie B. Gordon, Peabody, MA (US)

(73) Assignee: The Progeria Research Foundation, Peabody, MA (US)

(21) Appl. No.: 18/595,507

(22) Filed: Mar. 5, 2024

Related U.S. Application Data
(63) Continuation-in-part of application No. PCT/IB22/62870, filed on Dec. 29, 2022.
(60) Provisional application No. 63/294,418, filed on Dec. 29, 2021.
(60) Provisional application No. 63/488,502, filed on Mar. 5, 2023.

(51) Int. Cl. G01N 33/68

(52) U.S. Cl. CPC G01N 33/6893; G01N 2800/52

(57) ABSTRACT

Provided herein is a newly developed immunoassay that can detect progerin with high sensitivity, but which does not detect wildtype lamin A. Applications of the newly developed immunoassay in novel methods for diagnosing, prognosing, and treating progerin-related aging pathologies are also described.

### CROSS-REFERENCE TO RELATED APPLICATIONS

This is a continuation in part of International Patent Application No. PCT/IB2022/062870, filed on Dec. 29, 2022, which claims the benefit of U.S. Provisional Patent Application No. 63/294,418, filed on Dec. 29, 2021. Benefit is also claimed to U.S. Provisional Patent Application No. 63/488,502, filed Mar. 5, 2023. The contents of the foregoing patent applications are incorporated by reference herein in their entirety.

### ACKNOWLEDGMENT OF GOVERNMENT SUPPORT

This invention was made in part with government support under Grant No. 1U01FD006886-01 awarded by the U.S. Food and Drug Administration. The government has certain rights in the invention.

### FIELD

This disclosure relates to a newly developed immunoassay that can detect progerin with high sensitivity and specificity such that it differentiates progerin from wild type lamin A, and to novel methods for diagnosing, prognosing, and treating conditions related to increased concentrations of progerin in a sample from a subject.

### BACKGROUND

Hutchinson-Gilford progeria syndrome (HGPS) is an ultra-rare (prevalence 1 in 18-20 million living individuals), uniformly fatal premature aging disease. There is no sex, ethnic or regional bias. Morbidity includes failure to thrive, generalized lipodystrophy, alopecia, bone dysplasia, and progressive atherosclerosis resulting in death predominantly from heart failure at an average age of 14.6 years.

HGPS is a sporadic autosomal dominant disease caused by mutations in LMNA that produce a toxic splice variant of the nuclear membrane protein lamin A, called progerin (De Sandre-Giovannoli et al.; Eriksson et al.). Ninety percent of cases have classic HGPS, caused by a point mutation at c.1824 C>T; the remaining 10% have nonclassic HGPS, caused by single base mutations within the spliceosome recognition sequence of intron 11 (Gordon et al. 2018; Gordon et al. 2014). Progerin protein acts in a dominant negative fashion; it is the presence of progerin, and not the relative decrease in normal lamin A levels, that results in disease (Scaffidi et al.). In support, mice lacking lamin A exhibit a normal phenotype (Fong et al.). A dose effect of progerin is supported by murine studies showing graded disease severity and shortened lifespan in heterozygous vs. homozygous progerin-expressing mice as well as extension of lifespan in HGPS mice receiving progerin-targeted treatments (Benedicto et al.; Cabral et al.). Case reports have reported low and high progerin levels expressed by fibroblast cultures from mildly vs severely affected progeroid patients, respectively (Bar et al.; Hisama et al.).

Unlike lamin A, progerin lacks the cleavage site for a zinc metalloprotease, causing it to remain persistently farnesylated, and thereby incorporated as part of the nuclear lamina long-term, where it accumulates and exerts cell damage. In vitro, mouse models and human studies have demonstrated that phenotypes of HGPS are delayed and/or reversed by inhibiting farnesylation. Clinical trials for children with HGPS demonstrated that treatment with the farnesyltransferase inhibitor (FTI) lonafarnib had cardiovascular, bone, audiologic and weight benefit (Gordon et al., 2012), and was associated with increased survival of an estimated 2.5 years on average. Collectively, this evidence resulted in lonafarnib being the first-ever FDA-approved drug for treatment of HGPS.

There is currently no validated biomarker for HGPS. Since progerin is the disease-causing protein, it is the optimal biomarker candidate, and although progerin protein has been detected using immunofluorescence or ELISA in human autopsy tissue and skin biopsies, these methods are not sufficiently sensitive or quantitative for clinical trial utility, and tissue detection is not feasible for obtaining serial patient samples. Because of the previously acknowledged limitations of an immunoassay approach, an alternative to immunodetection has recently been developed using a targeted mass spectrometry approach (Camafeita et al.). However, its clinical utility may be also limited.

In addition to children and young adults diagnosed with HGPS, the presence of progerin has also been associated with aging in general.

The development of clinically relevant biologic markers of disease is critical to identifying and implementing effective treatments for children and young adults with HGPS. Moreover, in view of its association with aging in general and particularly with aging related conditions such as arteriosclerosis and atherosclerosis, the development of a quantitative assay for detecting progerin has clear implications beyond the HGPS context.

Validation of the progerin as a biomarker, and the development of an assay that directly quantifies the disease-causing protein, has the potential to significantly improve both preclinical and clinical trial assessments of new treatment approaches both for HGPS and also for aging-related conditions in general.

Thus, a continuing need exists for methods of detecting progerin in a sensitive, specific manner from biological materials that can be obtained longitudinally from subject, including but not limited to children with HGPS in order to evaluate disease state and treatment efficacy. Although ongoing work (see online at grantome.com/grant/NIH/U01-FD006886-01) has pursued the goal of validating progerin as a biomarker, it was not previously demonstrated that sensitive detection of progerin could be used for diagnostic or prognostic purposes, or was sufficiently sensitive to monitor patient changes in progerin in response to a given HGPS treatment.

### SUMMARY

Described herein is a method for prognosis of Hutchinson-Gilford progeria syndrome (HGPS) in a subject that includes the steps of: providing a sample from a subject; quantitating the concentration of progerin in in the sample with a quantitative immunoassay, wherein the quantitative immunoassay detects progerin but does not detect wildtype lamin A protein; and comparing the concentration of progerin in the sample with an HGPS-positive control, wherein a concentration of progerin in the sample that is below the concentration of progerin in the HGPS-positive control indicates an increased life expectancy in the subject.

In particular embodiments of the described method, the sample is a liquid sample selected from plasma, serum, urine, and saliva. In another embodiment, the sample is a protein sample derived from cells, such as cultured cells or cells from a subject. In such embodiments, the protein is isolated by standard methods of cell lysis and protein isolation. It will be appreciated that varying degrees of sample purity can be used in the described methods. Accordingly, for example, isolated protein from lysed cells need not be 100% purity, but can be significantly less so, such as 90%, 80%, 70%, 60%, or even less pure.

In other particular embodiments, the quantitative immunoassay for use in the described method includes the steps of: contacting the sample with a capture antibody that binds to both wildtype lamin A and progerin, resulting in a mixture of captured lamin A and captured progerin; separating the mixture of captured lamin A and captured progerin from the sample; contacting the mixture of captured lamin A and captured progerin with a progerin-specific detection antibody comprising a detectable label, thereby binding the captured progerin but not the captured lamin A with the detection antibody; separating the detectable label from the mixture with captured lamin A; and detecting the detectable label, and thereby quantitating the amount of progerin in the sample.

In particular embodiments of the described methods, the control is from the same subject at an earlier time point.

In some embodiments, the lower limit of quantitation of the quantitative immunoassay is 59 pg/ml. In other embodiments, the upper limit of quantitation of the quantitative immunoassay is 30,000 pg/ml. In still other embodiments, the range of quantitation of the quantitative immunoassay is 59 pg/ml-30,000 pg/ml.

In certain embodiments, the described methods include the further steps of repeating the method one or more times after the subject undergoes a treatment for HGPS, wherein a decrease, further decrease, or maintained decrease in progerin concentration following the treatment indicates increased life expectancy.

In particular embodiments, the decrease in progerin concentration is between 35-62%.

In still other embodiments, a greater decrease in progerin concentration indicates a greater increase in life expectancy.

Also described herein is a method for determining the efficacy of a treatment for Hutchinson-Gilford progeria syndrome (HGPS), that includes the steps of: providing a pre-treatment sample from a subject, that is taken prior to administration of a treatment for HGPS; determining the concentration of progerin in the pre-treatment sample with a quantitative immunoassay, wherein the quantitative immunoassay detects progerin but does not detect wildtype lamin A protein; providing a post-treatment sample from the subject, that is taken during or after administration of a treatment for HGPS; determining the concentration of progerin in the post-treatment sample with the quantitative immunoassay; and comparing the pre-treatment concentration of progerin with the post-treatment concentration of progerin, wherein a significant decrease in progerin concentration in the post-treatment sample indicates that the treatment is effective.

In particular embodiments, the pre-treatment sample and the post treatment sample are liquid samples selected from plasma, serum, urine, and saliva.

In other embodiments, the method further includes providing one or more additional post-treatment samples that are taken from the subject at time points subsequent to the initial post-treatment sample, and wherein a continued or further decrease in progerin concentration indicates continued efficacy of the treatment.

In particular embodiments, the quantitative immunoassay includes the steps of contacting the sample with a capture antibody that binds to both wildtype lamin A and progerin, resulting in a mixture of captured lamin A and captured progerin; separating the mixture of captured lamin A and captured progerin from the sample; contacting the mixture of captured lamin A and captured progerin with a progerin-specific detection antibody comprising a detectable label, thereby binding captured progerin but not captured lamin A with the detection antibody; separating the detectable label from the mixture with captured lamin A; detecting the detectable label, and thereby quantitating the amount of progerin in the sample.

In some embodiments, the lower limit of quantitation of the quantitative immunoassay is 59 pg/ml. In other embodiments, the upper limit of quantitation of the quantitative immunoassay is 30,000 pg/ml. In still other embodiments, the range of quantitation of the quantitative immunoassay is 59 pg/ml-30,000 pg/ml.

In particular embodiments of the described methods the treatment for HGPS includes a farnesyl transferase inhibitor, which in particular embodiments is lonafarnib.

Additionally described herein is a method for treatment of Hutchinson-Gilford progeria syndrome (HGPS) in a subject, that includes the steps of providing a sample from a subject; quantitating the concentration of progerin in in the sample with a quantitative immunoassay, wherein the quantitative immunoassay detects progerin but does not detect wildtype lamin A protein; comparing the concentration of progerin in the sample with an HGPS-positive control, wherein if the concentration of progerin in the sample indicates that the subject has HGPS; and if the subject has HGPS, administering to the subject a treatment for HGPS that lowers progerin concentration.

In some embodiments, the lower limit of quantitation of the quantitative immunoassay is 59 pg/ml. In other embodiments, the upper limit of quantitation of the quantitative immunoassay is 30,000 pg/ml. In still other embodiments, the range of quantitation of the quantitative immunoassay is 59 pg/ml-30,000 pg/ml.

In particular embodiments of the described methods the treatment for HGPS includes a farnesyl transferase inhibitor, which in particular embodiments is lonafarnib.

Further described herein are analogous methods of diagnosis, prognosis, treatment, and determining the treatment efficacy for a progerin-related aging pathology. The noted methods can be carried out similarly to those described in the context of HGPS patients, but within the more generalized context of a non-HGPS patient with or suspected of having a progerin-related aging pathology.

The foregoing and other objects, features, and advantages will become more apparent from the following detailed description, which proceeds with reference to the accompanying figures.

### BRIEF DESCRIPTION OF THE DRAWINGS

FIG. 1 is a schematic of the progerin-specific SMC Immunoassay. 1. Lamin A/C capture antibody is bound to magnetic microparticles (circle) at the heavy chain nonspecific region; 2. Progerin-containing solution is added to wells and progerin binds to Lamin A/C antibody capture antibody at antibody binding sites; 3. Fluorescently labeled progerin-specific detection antibody is added and binds to progerin; 4. Elution—chemical separation of progerin+Fl-progerin-specific antibody from magnetic microparticles; and 5. Single molecule counting using laser detection of fluorescent label (rectangle).

FIG. 2 shows analytical performance of the progerin assay. Goodness of curve fit was generated using back interpolation of progerin calibration curves generated over 10 consecutive assay runs. A. full range of quantification. B. low-end range of quantification.

FIG. 3 shows plasma progerin levels for nonHGPS patients and untreated clinical trial patients. Each symbol represents a unique individual patient level vs. age. Y axes are log scale. A. nonHGPS patients (N=69)=triangles B. Untreated HGPS patients (N=74)=circles displayed along with the same nonHGPS patients shown in panel A that fall within the same age range as the patients with HGPS=triangles.

FIG. 4 shows plasma progerin levels for clinical trial patients. On-site Trial center visit numbers and time on therapy vs. mean±SEM progerin. Trial visits to BCH occurred at various times post-visit 1 for different trials. All visit 1 patients were naïve to therapy. A. ProLon1, treated with lonafarnib (N=25). B. Triple trial patients treated with triple therapy from baseline to visit 5 (N=13), then switched to lonafarnib monotherapy thereafter (visits 6,7; N=10). C. ProLon2 lonafarnib monotherapy (N=26). D. Long-term continuous therapy (N=13). Top and bottom box edges represent the 75th and 25th interquartile (IQR) ranges, respectively. Horizontal lines and X within boxes represent medians and means, respectively. Lower and upper whiskers represent Q1−1.5×IQR and Q3+1.5×IQR. *p<0.05; **p<0.01, ***p<0.001, ****p<0.0001; NS, p>0.05. Each visit is compared with the connected visit.

FIG. 5 shows progerin level is associated with survival. Risk of death incrementally decreased with lower plasma progerin (p<0.0001) using time-dependent joint modeling. Change in mortality risk (y-axis) versus all values in range of observed progerin decrease (x-axis) from 0 to 65,000 pg/mL. Solid line is % decrease in risk of death; dashed lines=95% CI. N=74 subjects (9 untreated with single samples and 65 treated with untreated baseline plus multiple on-therapy samples).

FIG. 6 shows that progerin level is associated with survival (p=0.0008). Decreased risk of death with decreased plasma progerin. Time-dependent Cox Model using progerin to predict survival, adjusting for age and sex. Change in mortality risk (y-axis) versus all values in range of observed progerin decrease (x-axis) from 0 to 65,000 pg/mL. Solid line is % decrease in risk if death; dashed lines=95% CI. N=74 subjects (9 untreated with single samples and 65 treated with untreated baseline plus multiple on-therapy samples).

FIG. 7 shows that extent of increased life expectancy is incrementally associated with lower plasma progerin and longer duration of time on therapy. Increase in life expectancy (y-axis) versus length of treatment (x-axis) shown for 3 selected changes in plasma progerin (N=65 subjects with baseline and multiple on-therapy samples), using dynamic modeling based on cRMST.

### DETAILED DESCRIPTION

### Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.,” is derived from the Latin exempli gratia, and is used herein to indicate a non-limiting example. Thus, the abbreviation “e.g.,” is synonymous with the term “for example.” Consistent with their established definitions in the United States, the term “comprises” means “includes,” and the term “consists essentially of” or “consisting essentially of” indicates that the active component or step of the described composition or method includes only the expressly recited component or step. Further consistent with their established definitions in the United States, compositions that “comprise” a given component can also in other embodiments “consist essentially of” that component. Similarly, methods that “comprise” a given set of steps can also in other embodiments “consist essentially of” the expressly indicated set of steps.

In case of conflict, the present specification, including explanations of terms, will control. All materials, methods, and examples are illustrative and not intended to be limiting.

### Prognostic Methods for HGPS

The development of a clinically relevant biologic marker is critical to identifying and implementing effective treatments for children and young adults with HGPS. To date however, the identification of such a biomarker was impeded due to immunological methods that were not sufficiently sensitive or quantitative for clinical trial utility. Moreover, detection of such a biomarker in tissue was not feasible for obtaining serial patient samples. Because of the limitations of an immunoassay approach, an alternative to immunodetection was developed using targeted mass spectrometry (Camafeita et al.), but its clinical utility may be also limited. The studies described herein validate progerin as a quantitatable biomarker for HGPS, and demonstrate that progerin concentration can be used to directly assess new treatment approaches for this universally fatal disease. Moreover, the described studies also demonstrate plasma progerin's quantitative relationship to patient survival, enabling for the first time, quantitative methods for prognosing patient survival.

Accordingly, described herein is a newly developed immunoassay that selectively quantitates progerin in easy to obtain liquid samples (e.g., plasma, urine, and saliva) from a subject. The developed immunoassay has allowed for development of methods for prognosis of HGPS in a subject which can be used both to determine the efficacy of an HGPS treatment, and which also can be used to prognose a relative length in patient survival.

To determine efficacy of an HGPS treatment, the described methods involve providing a sample from a subject prior to undergoing the HGPS treatment; quantitating the concentration of progerin in this pre-treatment sample with a quantitative immunoassay; providing a sample that is taken from the subject after the subject undergoes the HGPS treatment; quantitating the concentration of progerin in in this post-treatment sample with the quantitative immunoassay; and comparing the concentration of progerin in the post-treatment sample with that of the pre-treatment sample. Based on this comparison, a concentration of progerin in the post-treatment sample that is significantly below the concentration of progerin in the pre-treatment sample indicates efficacy of the treatment.

In particular embodiments, the described method is used to quantitate progerin concentration in a sample taken from the subject at a single time point following administration of the treatment. In other embodiments, the described method can be used to quantitate progerin in longitudinal samples that are taken from the subject at successive time points following initial administration of the treatment and in particular embodiments, during continued administration of the treatment. In those methods in which progerin concentration is quantitated over multiple time points, a given treatment is deemed to be effective if there is a further decrease in progerin concentration from concentration of progerin following the initially measured time point, or if the decreased level of progerin is maintained over time.

As noted, embodiments of the described methods can be used to determine the relative length of HGPS patient survival. Such embodiments involve providing a sample from a subject; quantitating the concentration of progerin in the subject with a quantitative immunoassay; and comparing the concentration of progerin in the sample with a control concentration that is indicative of an HGPS-positive subject. Based on this comparison, a concentration of progerin in the subject that is lower than the concentration of progerin in the control indicates an increased life expectancy in the subject.

The samples for use in the described methods include any sample that may be obtained directly or indirectly from a subject, and from which cellular or extracellular protein can be detected. Such samples include but are not limited to whole blood, plasma, serum, tears, mucus, saliva, urine, sputum, tissues, cells (such as, fibroblasts, peripheral blood mononuclear cells, or muscle cells), organs, and/or extracts of tissues, cells (such as, fibroblasts, peripheral blood mononuclear cells, or muscle cells), or organs. A sample is collected or obtained using methods well known to those skilled in the art. In a particular embodiment, samples for use in the described methods include liquid samples such as plasma, serum, urine, or saliva. In still other particular embodiments, the sample taken from the subject and provided for use in the described methods is a plasma sample.

In the described methods, “providing” a sample includes any method known to the art for collecting and processing a sample from a subject for use in immunological methods of detecting protein. In particular embodiments, the sample is provided for the described immunoassay immediately after collection from the subject. In other embodiments, the sample is preserved, such as by freezing, for short and long-term storage of days, weeks, months, or years. Collection and preservation methods for protein-containing samples are standard and well known in the art. Similarly, in certain embodiments, progerin is detected using the described quantitative immunoassay without additional extensive preprocessing of the sample. Alternatively, the samples are processed sufficiently to allow for protein detection by the immunoassays described herein. Such protein extraction and concentration procedures are not necessary for all samples, but can be done as needed prior to providing the sample for the described methods in order to increase detected signal.

The prognostic methods described herein quantitate progerin by use of a quantitative immunoassay that selectively detects progerin in the sample(s) collected from the subject, but which does not detect the wildtype (non-mutant) form of the protein, lamin A. The quantitative immunoassay must be sufficiently sensitive to detect differences in progerin concentrations in a sample from sources of comparison (e.g., historic controls or from an earlier time point from the subject) in the picograms/ml range. For example, the quantitative immunoassay must be capable of detecting differences in 10,000 pg/ml, 5,000 pg/ml; 4,000 pg/ml, 4,000 pg/ml, 3,000 pg/ml, 2,000 pg/ml, 1,000 pg/ml, or less. In a particular embodiment, the lower limit of quantitation of the quantitative immunoassay is 59 pg/ml. In another embodiment, the upper limit of quantitation of the quantitative immunoassay is 30,000 pg/ml.

Immunoassays are well known to the art, and it will be appreciated that any immunoassay that can be adapted to specifically distinguish between wildtype and mutant variants of a protein, and which is sensitive enough to quantitate the target protein at the picogram scale as described, can be adapted for use in the described methods. Particular non-limiting examples of immunoassays that can be used in the described methods include radioimmunoassays (RIA), immunohistochemistry, immunofluorescent histochemistry with or without confocal microscopy, enzyme linked immunoabsorbance assays (ELISA) and related non-enzymatic techniques, and flow cytometry-related methodologies (see e.g. Cox et al., “Immunoassay Methods” in Assay Guidance Manual, Markossian et al., eds., Eli Lilly and Co., updated 2019). In a particular embodiment, the immunoassay is a typical “sandwich assay” or derivative thereof in which an antigen of interest is first captured from a sample by an antibody, and is then detected by a second antibody that specifically recognizes the antigen, and which is conjugated to a detectable label.

Immunoassays for use in the described methods employ antibodies that specifically recognize lamin A and progerin. Such antibodies can be monoclonal or polyclonal. Particular examples of antibodies for use in the described methods include intact immunoglobulins and the variants and portions thereof that are well known in the art, such as Fab′ fragments, F(ab)′2 fragments, single chain Fv proteins (“scFv”), and disulfide stabilized Fv proteins (“dsFv”).

More than specific structure or molecular arrangement, antibodies for use in the described method must be able to specifically bind to lamin A and progerin (capture antibodies) or bind specifically to progerin but not lamin A (detection antibodies). The terms “bind specifically” and “specific binding” refer to the ability of an antibody to bind to a target molecular species in preference to binding to other molecular species with which the specific binding agent and target molecular species are admixed. A specific binding agent is said specifically to recognize a target molecular species when it can bind specifically to that target.

The amino acid sequences of lamin A and progerin are known to the art (available online at NCBI accession numbers NP_733821.1 (lamin A) and AAR29466.1 (progerin)). Lamin A is a 664 amino acid polypeptide, and its truncated variant progerin is 614 amino acids. Accordingly, an antibody for use in the described methods, or fragment thereof, that specifically recognizes progerin but does not recognize lamin A is designed to recognize those C-terminal amino acids that are present in progerin, but not in lamin A. A particular non-limiting example of such an antibody recognizes an epitope between amino acids 604 and 611 of progerin, which cross the deletion region of lamin A. Antibodies for use in the described methods are commercially available.

The detection antibodies for use in the described methods are conjugated to a detectable label that includes any detectable compounds or compositions that can be conjugated directly or indirectly to an antibody that specifically recognizes progerin, but does not recognize or bind to lamin A. Specific, non-limiting examples of detectable labels for use in the described methods include radioactive isotopes, enzyme substrates, co-factors, ligands, chemiluminescent or fluorescent agents, haptens, and enzymes. Methods of detecting such labels are similarly standard.

In a particular embodiment the described quantitative immunoassay is a sandwich-type immunoassay that includes the steps of: (a) contacting the sample with a capture antibody that binds to both wildtype lamin A and progerin, resulting in a mixture of captured lamin A and captured progerin; (b) separating the mixture of captured lamin A and captured progerin from the sample; (c) contacting the mixture of captured lamin A and captured progerin with a progerin-specific detection antibody comprising a detectable label, thereby binding the captured progerin but not the captured lamin A with the detection antibody; (d) separating the detectable label from the mixture with captured lamin A; and (e) detecting the detectable label, and thereby quantitating the amount of progerin in the sample.

Various methods are known to the art for separating a given target protein from a complex mixture, as in step (b) of the above method. In particular embodiments, the capture antibody is attached to a solid substrate, such as, but not limited to magnetic particles, polystyrene beads, which can also be coated with an affinity tag, the well of a culture dish, and the like. Through the use of such substrates, the target protein (in this instance lamin A or progerin) is bound to the substrate once it is specifically recognized by and bound to the capture antibody. The captured antibody-protein-substrate can then be separated from the remaining sample components by standard methods and according to the nature of the substrate. For example, in particular embodiments wherein the capture antibody is coated on magnetic particles, the captured antibody-protein-substrate complex can be secured by way of a magnetic field while the remainder of the sample is washed away under sufficiently stringent conditions to prevent non-specific binding of protein to the antibody. In another exemplary embodiment, the capture antibody is affixed to the bottom of a culture plate, such as a 96-well culture plate, such that the captured antibody-protein complex will remain on the plate which the remainder of the sample can washed away.

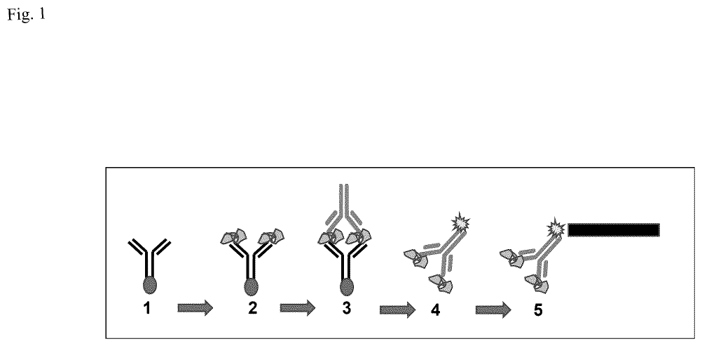

Similarly, following binding of the detection antibody to the captured antibody-protein complex (step (c), above), and excess detection antibody has been washed away under conditions that limit non-specific detection antibody binding, various methods of separating the bound detectable label (step (d)) are possible within the scope of the described method. In particular embodiments, the portion that is separated from the detection antibody-progerin-capture antibody complex is the detection antibody-progerin subcomplex (similar to the separation step 4 as depicted in FIG. 1). In other embodiments, it is only the antibody, along with its label, that is removed, while in still other embodiments, it is only the label that is separated from the bound complex. In particular embodiments, the label is separated (whether as part of the antibody-progerin, antibody, or apart from the antibody) by enzymatic means that cleaves one or more portions of the polypeptide complex. In other embodiments, it is separated by non-enzymatic means that release the label from the complex (whether as part of the antibody or antibody-progerin complex).

In determining the efficacy of a treatment for HGPS, the concentration of progerin is determined in a subject prior to administration of the treatment and after administration of the treatment. In a particular embodiment, efficacy is determined at a single time point following administration of the treatment. In other particular embodiments, efficacy is determined at multiple time points after or during administration of the treatment. For example, a sample can be obtained from a subject over a series of hours, days, weeks, months, and even years following administration of the treatment or during the continued course of the treatment. In a particular embodiment, a sample is collected from the subject every 2, 4, 6, 8, 10, 12, months and increments in between following or during administration of the treatment. In another embodiment, a sample is collected every half year or year during the course of the treatment or following its conclusion.

Efficacy of treatment for HGPS is indicated by the described methods in detecting a significant decrease in the concentration of HGPS following or during administration of the treatment. A significant decrease can be a statistically significant decrease, but in the current methods it does not need to be statistically significant decrease. Significant decreases in progerin concentration that indicate efficacy of a treatment include decreases from a detected pre-administration progerin concentration of up to 10%, at least 10%, at least 20%, at least 30%, at least 40%, at least 50%, at least 60%, at least 70%, or even greater. In particular embodiments, the decrease in progerin concentration is determined to be at least 35%-65% lower than the pre-administration progerin concentration.

In those embodiments in which samples are collected from the subject at multiple timepoints following the initial administration of a treatment, and progerin concentration is determined, efficacy of treatment can be determined either by a further decrease in progerin concentration as described, or by a maintenance of the reduced progerin concentration that is determined from the sample collected at the initial time point following administration of the treatment. It will be appreciated that in such embodiments, the further decrease in progerin concentration that is detected may be less than the initial decrease. Such further decreases can be as little as 1%-10%, but can also be more, as described above.

The methods for determining efficacy of a treatment for HGPS can be used to monitor the efficacy of any treatment that is expected to alter the expression or accumulation of progerin in the subject, and which can additionally be used in methods of treating a subject who, by detecting progerin using the described methods, is diagnosed with a progerin associated aging related disease.

Potential treatments for progerin associated aging related diseases such as but not limited to HGPS, and which aim to alter progerin levels using widely varying mechanisms of action have been or are under development (reviewed in Macicior et al.), including genetic editing (see e.g., Koblan et al. and International Patent Publication No. WO2020051360A1), Nucleic acid-based therapies (see e.g., Erdos et al.; Puttaraju et al.; International Patent Publication No. WO2017190041A1; and International Patent Publication No. WO2013086444A2), and small molecules that target progerin protein's post-translational processing, interactions with lamin A (e.g., progerinin as described in Kang et al.), and rate of autophagy. In a particular embodiment, the method monitors the efficacy of administering a farnesyl transferase inhibitor, such as lonafarnib to a subject with HGPS. The contents of the noted publications with respect to their descriptions of progeria treatments are incorporated by reference herein.

Particular embodiments of the disclosed methods are directed to prognosing relative life expectancy in a subject with HGPS. As described, lower concentrations of progerin in samples from a subject correlate with increased life expectancy. The described prognostic methods compare the concentration of progerin in a sample from a subject with that of an HGPS-positive control. As generally understood in art, a control is a reference standard. Accordingly, an HGPS-positive control can be a known or previously-determined progerin concentration in a subject or from a historical average. In particular examples an HGPS-positive control is the determined progerin concentration in a sample from the same subject (from whom the method sample is provided) at an earlier time point in their life, or at a time point prior to receiving an HGPS treatment. In other examples, an HGPS-positive control is the determined progerin concentration from one or more known other (non-subject) HGPS-positive individuals. In particular embodiments, an HGPS-positive control is the average concentration of progerin that is determined in samples from HGPS-positive subjects, such as recorded as a historic baseline for HGPS patients.

Accurate determination of progerin in clinical settings is a critical aspect for effective diagnosis, prognosis, and treatment of HGPS (see e.g., (Benedicto et al.; Cabral et al.). Using the described immunoassay, it can now be shown for the first time that a greater decrease in progerin concentration in the subject in comparison to an HGPS-positive control is quantitatively indicative of a greater relative increase in life expectancy. As with the methods of determining treatment efficacy described above the relative decrease need not be statistically significant, but can be as described above, decreases from a control can be up to 10%, at least 10%, at least 20%, at least 30%, at least 40%, at least 50%, at least 60%, at least 70%, or even greater. In particular embodiments, the decrease in progerin concentration is determined to be at least 35%-65% lower than the control. Increases in projected lifespan.

Similar to the methods for determining efficacy of an HGPS treatment, in particular embodiments, the prognostic methods described herein can be employed at a single time point after a subject (patient) is diagnosed. In other embodiments, the methods described herein can be used to monitor the progress of a patient and any changes in progerin concentration over time. Multiple time points can be used in such monitoring, for example, months, years, or more after diagnosis and treatment initiation (and any time point in between) can be suitable timepoints to measure the concentration of progerin in a sample from the patient.

### Methods for Treatment of HGPS

In the current disclosure, progerin is validated as a biomarker for HGPS. It is shown not only that patients diagnosed with HGPS have significantly increased concentrations of progerin, but also that progerin concentrations are significantly decreased in patients who receive certain treatments for HGPS, and that decreases in progerin concentration correlate with increases in life expectancy. Accordingly, described herein are methods for treatment of a subject with HGPS. In such methods, a sample is provided from a subject who may be suspected of having HGPS or who may not be suspected of having HGPS. The concentration of progerin in the sample is determined as described above, and is compared with an HGPS-positive control, which can be a progerin concentration from one or more individuals known to have HGPS. If the concentration of progerin in the sample indicates that the subject has HGPS (e.g., because the concentration of progerin is similar, equal to, or greater than the concentration of progerin in the HGPS-positive control), then the subject is provided a treatment for HGPS that lowers progerin concentration.

As noted, multiple treatments for HGPS are under development to alter progerin levels using widely varying mechanisms of action. Any of the noted treatments, if validated, can be used in the current methods. To date, the only approved method of treatment is use of the farnesyl transferase inhibitor lonafarnib.

Farnesyltransferase inhibitors (FTIs) are a class of compounds which inhibit the ability of farnesyltransferase to transfer a farnesyl group from farnesyl pyrophosphate to a given target protein. FTIs can be used to inhibit the addition of farnesyl to progerin, thereby preventing the aberrant association of progerin or prelamin A with the inner nuclear envelope. FTIs generally can be divided into three groups: (1) tetrapeptides having or mimicking the CAAX motif (Brown et al., Proc. Natl. Acad. Sci. U.S.A. 89:8313-8316, 1992; Reiss et al., Proc. Natl. Acad. Sci. U.S.A. 88:732-736, 1991; Goldstein et al., J. Biol. Chem. 266:15575-15578, 1991); (2) analogs of farnesyl pyrophosphate (FPP) (Gibbs et al., J. Biol. Chem 268:7617-7620, 1993); and (3) inhibitors with structures not resembling either tetrapeptides or FPP (Liu et al., J. Antibiot. 45:454-457, 1992; Miura et al., FEBS Lett. 318:88-90, 1993; Omura et al., J. Antibiot. 46:222-228, 1993; Van Der Pyl et al. J. Antibiot. 45:1802-1805, 1992). The latter category of inhibitors generally has lower activity compared to the first two categories. By way of example, the FTI lonafarnib (Zokinvy®) is a non-peptidomimetic FTI; FTI-277 is a peptidomimetic. Another non-limiting example of a FTI for use in the methods described herein include R115777 (tipifarnib, Zarnestra®). The development and chemistry of FTIs are well documented and known to those of ordinary skill. Although to date, lonafarnib is the only FTI approved for use in treatment of HGPS, It is believed that all categories of FTIs can be used in the methods provided herein; and the selection of a specific FTI is within the skill of the ordinary practitioner. In some embodiments, it is beneficial to select an inhibitor compound that is more selective for farnesyltransferase, compared to geranylgeranyltransferase I. In other embodiments, it may be beneficial to select an inhibitor compound that is dually selective, in that it inhibits both FTase and GGTase I. Considerations for determining selectivity criteria for FTIs include (but are not limited to) the possibility of lower toxicity with FTase-specific FTIs versus dual specificity FTIs, although both efficacy and toxicity may differ according to the particular compound and the particular patient. As will be recognized by an ordinarily skilled practitioner, other considerations, for instance pharmacological and medical considerations, may also apply.

In particular examples, the FTI, such as lonafarnib is administered at a dosage range between 115 mg/m² to 150 mg/m²/day, such as about 115 mg/m², 120 mg/m², 125 mg/m², 130 mg/m², 135 mg/m², 140 mg/m², 145 mg/m², 150 mg/m². One of skill will appreciate that other dosage ranges typical for FTIs are encompassed by this disclosure.

The treatments for use in the methods described herein typically are contained in a pharmaceutically acceptable carrier and/or pharmaceutical excipient. The term “pharmaceutically acceptable” means approved by a regulatory agency of the federal or a state government or listed in the U.S. Pharmacopoeia or other generally recognized pharmacopoeia for use in animals, and, more particularly, in humans. The term “carrier” refers to a diluent, adjuvant, excipient, or vehicle with which the therapeutic is administered.

Examples of pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Particular treatments, as appropriate, can also contain minor amounts of wetting or emulsifying agents, or pH buffering agents. Treatments for use in the methods described herein can take the form of solutions, suspensions, emulsion, tablets, pills, capsules, powders, sustained-release formulations, and the like. Oral formulations can include standard carriers such as pharmaceutical grades of mannitol, lactose, starch, magnesium stearate, sodium saccharine, cellulose, magnesium carbonate, and the like.

Embodiments of other pharmaceutical compositions are prepared with conventional pharmaceutically acceptable counter-ions, as would be known to those of skill in the art.

Therapeutic preparations will contain a therapeutically effective amount of at least one active ingredient, preferably in purified form, together with a suitable amount of carrier so as to provide proper administration to the patient. The formulation should suit the mode of administration.

The combination treatments of this disclosure can be formulated in accordance with routine procedures as pharmaceutical compositions adapted for intravenous administration to human beings. Typically, compositions for intravenous administration are solutions in sterile isotonic aqueous buffer. Where necessary, the compositions may also include a solubilizing agent and a local anesthetic such as lidocaine to ease pain at the site of the injection.

The ingredients in various embodiments are supplied either separately or mixed together in unit dosage form, for example, in solid, semi-solid and liquid dosage forms such as tablets, pills, powders, liquid solutions, or suspensions, or as a dry lyophilized powder or water free concentrate in a hermetically sealed container such as an ampoule or sachette indicating the quantity of active agent. Where one or more of the indicated agents is to be administered by infusion, it can be dispensed with an infusion bottle containing sterile pharmaceutical grade water or saline.

The specific dose level and frequency of dosage for any particular subject may be varied and will depend upon a variety of factors, including the activity of the specific compound, the metabolic stability and length of action of that compound, the age, body weight, general health, sex, diet, mode and time of administration, rate of excretion, drug combination, and severity of the condition of the host undergoing therapy.

The therapeutic compounds and compositions of the present disclosure can be administered at about the same dose throughout a treatment period, in an escalating dose regimen, or in a loading-dose regime (e.g., in which the loading dose is about two to five times the maintenance dose). In some embodiments, the dose is varied during the course of a treatment based on the condition of the subject being treated, the severity of the disease or condition, the apparent response to the therapy, and/or other factors as judged by one of ordinary skill in the art. In some embodiments long-term treatment with the drug is contemplated.

In some embodiments, sustained localized release of the pharmaceutical preparation that comprises a therapeutically effective amount of a therapeutic compound or composition may be beneficial. Slow-release formulations are known to those of ordinary skill in the art. By way of example, polymers such as bis(p-carboxyphenoxy)propane-sebacic-acid or lecithin suspensions may be used to provide sustained localized release.

### Progerin-Related Aging Pathologies

Progerin is associated with aging in general, and particularly with aging pathologies such as cardiovascular diseases. It will therefore be appreciated that the quantitative immunoassay described herein is applicable to the broader context of generalized aging. Accordingly, the methods for diagnosis, prognosis, treatment, and assaying treatment efficacy that are described herein with respect to HGPS are also applicable to diagnosis, prognosis, treatment, and assaying treatment efficacy of progerin-related aging pathologies in general. Particular non-limiting examples of progerin-related aging pathologies include cardiovascular diseases such as arteriosclerosis and atherosclerosis, insulin resistance (pre-diabetes), alopecia, and lipodystrophy.

In addition, in view of the association between progerin and aging, the described assay could be used as a way to monitor aging and overall subject health. For example, progerin could be lower in long-lived, comparatively healthy individuals, with lower levels of progerin indicating a maintenance of health in long-lived individuals.

As noted, particular embodiments of the claimed methods include determining the prognosis of a patient with a progerin-related aging pathology by quantitating the amount of progerin in a sample from the patient and comparing the measured amount to one or more controls. In particular embodiments, the concentration of progerin that is quantitated in the patient is greater than the control, and can indicate a negative prognostic outlook for the course of the pathology. In other embodiments, the concentration of progerin that is quantitated in the patient is lower than the control, thereby indicating a positive prognostic outlook for the patient. Although “negative” and “positive” prognostic indications will be generally understood by one of skill in the art to indicate respectively a worsening and improvement in the course of the pathology. For example a negative prognostic outlook for a patient with arteriosclerosis can be increased blood pressure, and increased risk of severe cardiovascular complications. Conversely, a positive prognostic outlook for such a patient can be a decrease in a previously elevated blood pressure, and an associated decreased risk of severe cardiovascular complications.

The following examples are provided to illustrate certain particular features and/or embodiments. These examples should not be construed to limit the disclosure to the particular features or embodiments described. Portions of the following examples were also published in Gordon L B et al., Circulation. 2023; 147:1734-1744, the contents of which are incorporated by reference herein in their entirety.

### Examples

### Example 1: Methods: Single Molecule Counting (SMC™) Progerin Immunoassay

An SMC ultrasensitive immunoassay for the detection of plasma progerin was developed. Overall, the assay employed a sandwich immunoassay format with an anti-lamin A capture antibody coated on magnetic microparticles, and an anti-progerin specific detection antibody conjugated to a fluorescent tag (FIG. 1) run in a 96-well plate. Progerin-bound detection antibody was eluted and read as single molecules using a confocal laser.

The SMC assay for measuring Progerin was as previously described (Todd et al.), except that magnetic microparticles (MP) coated with capture antibodies and detection reagents were specific to the progerin assay (FIG. 1). Briefly, the progerin capture antibody (mouse Mab anti-Lamin A+C, clone: 131C3 Abcam, epitope between amino acid residues 319-566, common to Lamins A, C and progerin) was coated onto MP at 25 μg antibody/mg MP. Samples were exposed to the MP-capture antibody, to specifically bind analyte, followed by one plate wash to remove unbound material. Next, fluorescently labeled progerin-specific detection antibody (Mouse Mab, clone: 13A4, from Millipore, epitope between amino acids 604 and 611 of progerin, which cross the deletion region of lamin A), pre-diluted to 2,000 ng/mL in assay buffer and then filtered through a 0.2 μm filter (MilliporeSigma), was added to wells. Plates were then washed 4 times to remove any unbound detection antibody. All MPs were then transferred from the initial 96-well assay plate to a new plate to avoid eluting any non-specific plate-bound detection antibody. Progerin-bound detection antibody was then eluted by pH shift using a proprietary glycine buffer, transferred to a new plate and detected by SMC using the Erenna Immunoassay System software (SgxLink™). The raw signal data and the interpolated value data obtained from SgxLink were transferred to Excel where precision was then calculated for the replicate wells. The acceptance specification for raw signal and interpolated values was precision and recovery bias≤20%.

A 12-point standard curve of recombinant progerin (Abcam 93918) diluted in EMD standard diluent, a synthetic serum-based solution containing a proprietary mixture of Tris buffer and carrier protein (emdmillipore.com), was run on each plate in triplicate. The standard curve was used to determine progerin concentration in unknown samples. Samples were diluted in standard diluent so that anticipated progerin fell within the dynamic range of the standard curve. All reported progerin and SD are dilution-corrected. All samples were tested in duplicate, unless triplicate is specified.

Signal at or below the background plus two standard deviations was reported as not detected (ND). The lower limit of quantification (LLoQ) was prospectively defined as the lowest point on the standard curve, from the top down, which was recovered within 20% of expected and had a % coefficient of variation (CV) of ≤20%.

To assure similar progerin quantitation between assays at high, mid- and low values, the same set of endogenous plasma inter-assay quality controls (QC) were assayed on each plate in duplicate. Endogenous plasma progerin was derived from a homozygous transgenic mouse model of HGPS that expresses the human LMNA gene harboring the classic pG608G, c.1824 C>T mutation (Murtada et al.). This mouse plasma contains high levels of progerin and allowed QC generation without utilizing precious human HGPS plasma stores. Aliquots were diluted in normal human plasma for storage at −80° C. QC plate acceptability parameters were locked in as mean±20% of the first 24 QC runs during assay development. The high and mid-QCs mean progerin was 188,206±10,580 pg/mL (% CV=5.6) and 19,513±1,235 pg/mL (% CV=6.3), respectively. The low QC was always below the LLOQ. To mirror the 1:25 dilution protocol for clinical trial samples, QCs were filtered and diluted 25-fold on the same day as the assay was performed. Average in assay progerin for high and mid-QCs in clinical trial sample plates (N=39) were 7,303±609 pg/mL (% CV=3.8) and 755±90 pg/mL (% CV=5.7), respectively. Thus, high- and mid-QC always fell within the analytical measurement range.

A valid run required the lower limit of quantitation calculated from the standard curve to be ≤59 pg/mL, and the controls to be within 20% of the established high and medium QCs.

Once final assay conditions were established, samples with interpolated concentrations that were above the upper limit of quantitation (ULoQ), or those with % CV>20% were re-tested using appropriate dilution to assure progerin levels fell within the limits of quantitation of the assay. In addition, any plate in which the standard curve LLoQ was not met (599 pg/mL) or QCs (high and medium, with low control falling below LLOQ) fell outside of 20% of the mean and SD was repeated.

### Dilutional Linearity

Linearity of dilution was calculated by dividing the dilution corrected progerin value in the dilution series by its preceding dilution's progerin value and expressed as a percentage:

$$DL = {[ obs ]}_{B} {/ [ obs ]}_{A} * 100 , \\ where B is the dilution corrected value of the higher dilution and \\ A is the dilution corrected value at the previous dilution level .$$

### Freeze-Thaw Specimen Stability

Plasma samples frozen at −80° C. were thawed at room temperature (RT) for 15 minutes, mixed well, and aliquoted to create 3 additional aliquots for this study. All aliquots were then refrozen at −80° C. prior to being subjected to additional freeze-thaw testing. On the day of testing, assigned aliquots were thawed to room temperature for 15 minutes and refrozen at −80° C. for up to 4 freeze-thaw cycles. The final thawed samples were then diluted and assayed in triplicate. All samples were tested on the same day. The freeze-thaw sample stability was acceptable if the % difference at freeze-thaw cycle 2 and 5 was ±20% (industry standard).

The percent difference (% Diff) from Cycle 2 of each sample at freeze-thaw cycles 3, 4, and 5 was calculated using the following formula:%

$$Diff = \frac{( Mean C cycle \# 2 - 4 ( pg / mL ) - Mean C cycle \# 1 ( pg / mL )}{Mean C cycle \# 1 ( pg / mL )} \times 100$$

Detection of Native Progerin vs. Recombinant Progerin

Healthy nonHGPS plasma samples (commercially purchased) were diluted 50-fold in standard diluent and then spiked with either a 10% by volume standard diluent, recombinant progerin yielding a 1,000 pg/mL, or HGP1110 designed to deliver 1,000 pg/mL progerin. The actual calculated progerin spike was determined from the buffer spiked sample HGP1110 control value and was 1,019 pg/mL.

$$Spike recovery ( \% ) = ( Spiked sample progerin ( pg / mL ) - endogenous progerin ( pg / mL ) / expected progerin spike ( pg / mL ) .$$

### Lonafarnib Interference Assay

Lonafarnib API (Batch #MK-6336-000R025) was obtained from The Progeria Research Foundation, Inc. (PRF) Cell and Tissue Bank. HGPS plasma was spiked with 2 μL of either dimethyl sulfoxide (DMSO) or lonafarnib dissolved in DMSO 7.92 μg/mL. Percent (%) interference=[(mean concentration of spiked sample−mean concentration of control)/mean concentration of control]×100.

### Human Clinical Data and Samples

Human clinical data was obtained from The PRF International Progeria Registry, Diagnostics Program, and Medical and Research Database (available online at progeriaresearch.org). HGPS plasma and serum were obtained from The PRF Cell and Tissue Bank (available online at progeriaresearch.org). Clinical trial samples were donated to the Bank from Boston Children's Hospital (BCH) with informed consent or approval from the BCH Committee on Clinical Investigation. Trial samples were collected in a fasting state, in the morning just prior to treatment dosing as trough samples, except pK samples which were collected at trough and then at specified times post-trough. Non-trial samples were not regulated as to time of day collected. Some nonHGPS healthy human plasma samples were obtained from ProMedDx. HGPS and nonHGPS plasma sample IDs were assigned HGPI and NPI prefixes, respectively.

The nonHGPS study cohort consisted of patients who tested negative for suspected HGPS or their relatives. Plasma from nonHGPS patients with stage 2 or 3 kidney disease and congestive heart failure, and healthy controls used for assay development were purchased from Precision for Medicine (Norton, MA).

Patients with HGPS had genetically confirmed progerin-producing mutations in the LMNA gene. Samples from patients with classic HGPS (c.1824 C>T) were presented in the main analyses, while nonclassic HGPS comparisons were used in subgroup comparisons.

For plasma isolation, blood was collected into either sodium heparin (either heparin or CPT™ vacutainers) and centrifuged at 4° C. for 15 minutes at 1500×g, or K₂EDTA and centrifuged at 1300 g for 10 min at room temperature (RT) (all BD Biosciences). For serum isolation, blood was allowed to clot at room temperature for 60 minutes (±10 minutes) and then centrifuged at RT for 10 minutes at 1100×g. All samples were aliquoted, stored at −80° C. and sent to EMD Millipore on dry ice for analysis.

For analysis, samples were fully thawed at RT for <5 min, lightly mixed, diluted in standard diluent before transferring into filter plate wells (MilliporeSigma MSBVN1210). Patient plasma volumes required for analysis were 200 or 100 μl (nonHGPS 1:1 and 1:2) and 8 μl (HGPS 1:25) to generate duplicates. Filter plates with sample were centrifuged for 10 minutes to pull sample through the filters. Filtrate was then assessed as described.

### Study Drug Dosing and Administration

Clinical trial study samples are separated into 3 categories, with acronyms ProLon1 (Gordon et al., 2014; Gordon et al., 2012), Triple Therapy (Gordon et al., 2016) and ProLon2 (Gordon et al., 2018), which were conducted sequentially as open label single center trials at Boston Children's Hospital (see previously published study details). In all clinical trials, patients received oral lonafarnib (Schering-Plough Research Institute or Merck&Co., Inc., or Eiger Biopharmaceuticals) either by capsule or liquid suspension, every 12±2 hours. Lonafarnib dosing was 150 mg/m², except for the first 4 months of ProLon1 when dosing was 115 mg/m².

For Triple Therapy Trial oral pravastatin (Pravachol, Bristol-Meyers Squibb) and zoledronic acid (Zometa, Novartis, Inc.) were administered along with lonafarnib.

### Sample Blinding and Inclusion Criteria

Once the assay development was established, all samples were assessed in a blinded fashion by EMD Millipore technical staff. Repeat samples were assigned differing, random ID numbers. ID assignments without HG or nonHG prefixes were submitted for analysis and converted to HG and nonHG identifiers.

For inclusion in longitudinal analyses, sample sets required a baseline and end-of-trial on-therapy plasma sample. To compare lonafarnib doses, ProLon1 inclusion required a baseline pre-therapy, 4-month (dose 115 mg/m²), and end-of-study sample (dose 150 mg/m²). For inclusion in pK analyses, sample sets required at least one trough and one post-trough plasma sample.

### Statistical Analyses

Data summaries are primarily descriptive; comparisons between sample sets utilized paired student's T tests, two sample T tests, and Pearson correlations. Descriptive statistics included sample size, mean and standard deviation. Statistical significance level was set at a two-sided p<0.05. There was no adjustment for multiple comparisons.

To assess the relationship between plasma progerin concentration and survival, two analyses were performed, both adjusting for age at baseline and sex. The first implemented a joint model with a time-dependent slopes parameterization, which assessed the effect of the current value of progerin on survival, adjusting for the change in progerin. The second implemented a time-dependent Cox model, treating progerin as a time-dependent covariate. Assessment of only baseline progerin on survival outcome was not conducted due to bias, since immediately after baseline, patients initiated treatment that was anticipated to affect progerin levels.

To quantify increase in life expectancy as it related to decrease in progerin and duration of time on lonafarnib therapy, the methodology by Yang et al. (2021), which proposes a dynamic model that utilizes a series of landmark time points (Dafni, 2011) based on the conditional restricted mean survival time (cRMST), was utilized. This analysis also adjusted for sex and age at baseline.

Excel was used for descriptive statistics. SAS version 9.4 was used for the time-dependent Cox model, the JM and dynpred packages in R version 4.0.2 were used for the joint modeling and cRMST dynamic model, respectively.

### Example 2: HGPS-specific Plasma Progerin Assay

This example describes the development and validation of an immunoassay for specific and sensitive detection of progerin in plasma samples.

### Development and Validation of Quantitative Assay for Plasma Progerin Detection

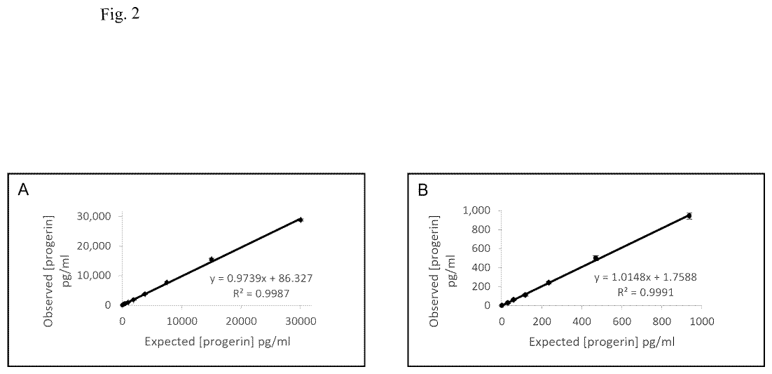

Standard Curve and Reporting Range: To develop an SMC progerin immunoassay standard curve and determine a reliable reporting range, recombinant progerin was spiked into buffer to achieve 30 ng/mL and serially diluted in steps of two-fold to generate a standard curve ranging from 30,000 to 29 pg/mL (FIG. 2, panels A and B). Signal was interpolated by SGXLink software. The dynamic detection range was 59 pg/mL (LLoQ)-30,000 pg/mL (ULOQ). The mean value of the (measured concentration)/(expected concentration) was 100% (range 95%-106), and a linear response (R²-0.9987) was observed from 59 pg/mL to 30,000 pg/mL. Consequently, all samples were diluted in standard diluent to accommodate this range. Specifically, nonHGPS samples were assessed at 1:1 or 1:2; HGPS samples were below the ULoQ when diluted 1:25.

Dilutional Linearity: To assess the assay's ability to consistently measure plasma progerin with varying sample dilutions in HGPS, human HGPS plasma samples (N=3) were quantified in triplicate at 5, 25, and 125-fold dilution in assay buffer (Table 1). All patient samples were quantifiable at all the dilutions tested and ranged in final progerin from 15,835 pg/mL to 50,610 pg/mL. Mean linearity of dilution was 98.2% (range 92-110). NonHGPS plasma (N=4) was assessed for linearity of 1:2 versus neat, to stay above the LLOQ.

TABLE 1
Dilutional Linearity*

| | | | | | Dilution | |
|---|---|---|---|---|---|---|
| | | Uncorrected | | | corrected | % of |
| Sample | | progerin | | % | progerin | lowest |
| ID | Dilution | pg/mL | SD | CV | pg/mL | dilution** |
| HGPl154 | 1:5 | 3,299.71 | 302.97 | 9 | 16,499 | NA |
| | 1:25 | 637.34 | 21.62 | 3 | 15,933 | 97 |
| | 1:125 | 120.55 | 5.38 | 4 | 15,069 | 95 |
| HGPl145 | 1:5 | 10,683.89 | 496.82 | 5 | 53,419 | NA |
| | 1:25 | 2,055.03 | 29.25 | 1 | 51,376 | 96 |
| | 1:125 | 376.28 | 34.63 | 9 | 47,035 | 92 |
| HGPl110 | 1:5 | 7,592.57 | 362.48 | 5 | 37,963 | NA |
| | 1:25 | 1,499.55 | 44.53 | 3 | 37,489 | 99 |
| | 1:125 | 328.47 | 22.40 | 7 | 41,059 | 110 |
| NPl1 | 1:1 | 244 | 2.47 | 1 | 244 | NA |
| | 1:2 | 101 | 5.78 | 6 | 201 | 83 |
| NPl2 | 1:1 | 264 | 12.04 | 2 | 264 | NA |
| | 1:2 | 134 | 13.19 | 10 | 268 | 102 |
| NPl3 | 1:1 | 115 | 0.77 | 1 | 115 | NA |
| | 1:2 | 50 | 5.30 | 11 | 100 | 87 |
| NPl4 | 1:1 | 282 | 5.14 | 2 | 282 | NA |
| | 1:2 | 95 | 7.24 | 8 | 189 | 67 |

*HGPS (HGPl) and nonHGPS (NP) samples were assayed at different dilutions to assess linearity of dilution. Results are presented as a percentage of the progerin concentration of the lowest dilution for each sample tested.
**NA = not applicable

Intra-assay and Inter-assay Variability: The precision of the SMC Progerin Immunoassay to reliably quantitate plasma samples within run (intra-assay) and day to day (inter-assay), with multiple replicates on multiple days was determined. Intra-assay variability (Table 2) demonstrated average CV of 7% (range 1-16), while inter-assay variability (Table 3) demonstrated average CV of 12% (range 7-18). In addition, excellent intra-assay precision was demonstrated from high- and mid-QCs, which were run on each clinical trial sample plate (N=39) in duplicate. Average dilution-corrected high-QC was 182,593±15,229 pg/mL progerin with 3.8% CV; and mid-QC was 18,880±2,260 pg/mL progerin with 5.8% CV.

TABLE 2
Plasma Progerin Intra-assay Variability*

| | replicate 1 | replicate 2 | replicate 3 | Mean | | |
|---|---|---|---|---|---|---|
| Sample | progerin | progerin | progerin | progerin | | % |
| ID | pg/mL | pg/mL | pg/mL | pg/mL | SD | CV |
| HGPl095 | 24,619 | 19,870 | 21,194 | 21,894 | 2,450 | 11 |
| HGPl092 | 39,850 | 37,152 | 37,440 | 38,130 | 1,495 | 4 |
| HGPl039 | 44,958 | 46,445 | 44,615 | 45,339 | 975 | 2 |
| HGPl110 | 29,967 | 30,053 | 30,302 | 30,107 | 175 | 1 |
| HGPl132 | 76,216 | 85,654 | 84,322 | 82,063 | 5,108 | 6 |
| HGPl145 | 36,658 | 30,422 | 26,912 | 31,330 | 4,935 | 16 |
| HGPl150 | 45,305 | 40,159 | 45,431 | 43,631 | 3,009 | 7 |
| HGPl154 | 16,682 | 13,868 | 14,289 | 14,946 | 1,520 | 10 |

*Eight different HGPS plasma samples were assayed in triplicate wells on the same assay plate to assess inter-assay variability. Dilution factor for all samples was 1:50.

TABLE 3
Plasma Progerin Inter-assay Variability**

| | plate 1 | plate 2 | plate 3 | plate 4 | plate 5 | plate 6 | Mean | | |
|---|---|---|---|---|---|---|---|---|---|
| Sample ID | pg/mL | pg/mL | pg/mL | pg/mL | pg/mL | pg/mL | pg/mL | SD | % CV |
| HGPl095 | 21,894 | 23,143 | 21,395 | 20,537 | 28,427 | 28,817 | 24,036 | 3,653 | 15 |
| * HGPl092 | 38,130 | 30,751 | 33,000 | 32,209 | 36,823 | 32,598 | 33,919 | 2,888 | 9 |
| HGPl039 | 45,339 | 44,898 | 32,246 | 30,339 | 41,586 | 41,019 | 39,238 | 6,419 | 16 |
| HGPl110 | 30,107 | 30,803 | 21,607 | 20,721 | 32,104 | 28,071 | 27,236 | 4,888 | 18 |
| * HGPl132 | 82,064 | 59,555 | 73,036 | 71,949 | 74,650 | 69,186 | 71,740 | 7,373 | 10 |
| HGPl145 | 31,330 | 31,932 | 28,455 | 26,866 | 33,499 | 33,522 | 30,934 | 2,724 | 9 |
| * HGPl150 | 43,632 | 36,368 | 39,106 | 40,285 | 43,424 | 41,094 | 40,651 | 2,744 | 7 |
| HGPl154 | 14,946 | 16,556 | 16,058 | 15,830 | 19,103 | 20,066 | 17,093 | 2,022 | 12 |

* These samples were repeated due to initial technical error during first series of plates.
**Dilution factor for all samples was 1:50; each sample run in triplicate in each of 6 assays. The average of each triplicate on a given plate is listed, followed by mean, SD and % CV for each sample ID.

### HGPS-Specific Plasma Progerin Assay Assessments: Lamin a Cross-reactivity and Sample Freeze-thaw Integrity

Progerin Assay Does Not Cross-React with Lamin A: To assess potential cross-reactivity between progerin and lamin A detection in the assay, two, twelve-point sample curves, one containing human recombinant lamin A and another containing human recombinant progerin were run in triplicate in the SMC Progerin Immunoassay starting at 10,000 pg/mL and 2-fold serially diluting to 9.8 pg/mL with a zero pg/mL buffer-only anchor point on each curve. The assay had no detectable cross reactivity to lamin A. Progerin recovery range was 97-111% of expected at the different concentrations tested, with R²=0.9991. Thus, no signal was generated by the presence of lamin A that competed or interfered with the quantification of progerin.

Spike Recovery and Detection of Native Progerin vs. Recombinant Progerin: To evaluate the assay's ability to measure recombinant progerin in comparison to native progerin, either was spiked into 3 different healthy nonHGPS donor plasma samples that were diluted 50-fold with standard diluent before receiving a spike. Both native and recombinant progerin yielded similar progerin recovery in the expected range, with average recoveries of 96±6 and 120±15%, respectively (Table 4).

TABLE 4
Detection of native progerin vs. recombinant progerin

| | | NonHGPS |
|---|---|---|
| | nonHGPS | Plasma + |
| | Plasma + | Recombinant |
| | HGPS Plasma | Progerin |
| | (N = 3) | (N = 3) |
| Spiked concentration | 1,019 | 1,000 |
| Expected concentration = | 1,051 | 1,032 |
| healthy plasma* + spike | | |
| Measured concentration | 1,014 ± 64 | 1,243 ± 153 |
| Average % recovery | 96 ± 6 | 120 ± 15 |

*NonHGPS plasma + buffer concentration (N = 3) = 32.4 ± 33.3 pg/mL

Freeze-thaw Stability: Samples presented in this study have been frozen prior to analysis. It is anticipated that only pre-frozen samples will be assessed routinely in clinical trials due to off-site assay execution. A freeze-thaw analysis of 4 freeze-thaw cycles demonstrated no differences between progerin (all p<0.05).

### Classic vs Nonclassic Genotypes

Both classic and nonclassic forms of HGPS are progerin-producing; classic due to optimization of an internal splice site and nonclassic due to de-optimization of the canonical splice site. Data from children with the classic genotype (G608G, c.1824 C>T; N=74) was compared with nonclassic HGPS patient samples (LMNA c.1822G>A, p.G608S×2, c.1968+1 G>A, c.1968+2 T>A, c.1968+5 G>C) (N=5; mean±SD=28,402±11,601 pg/mL). When comparing baseline off-therapy progerin to classic patient samples (N=74; mean±SD=33,249±12,299 pg/mL), all nonclassic patient progerin levels fell within the range of classic patient levels, and there was no overall difference detected (p=0.39). Similarly, baseline to end-of-study decreases in progerin with therapy (40.3-57.5%) were within the range of classic patients, and end-of-study values were not different from classic patients (p=0.85).

### Similarity in Progerin Quantitation Between NaHeparin-Isolated Plasma, K₂EDTA-isolated Plasma, and Serum

To assess whether the assay was suitable for progerin detection across different blood collection tube types, a comparative analysis of blood collected into tubes containing sodium heparin (plasma), K₂EDTA (plasma), and no anticoagulant (serum), from the same patient in the same blood draw (N=3 patients) was performed in triplicate. There were no significant differences between these 3 sample types; NaHeparin plasma vs. EDTA plasma p=0.98; NaHeparin plasma vs serum p=1.00; and EDTA vs serum p=0.97.

### Plasma Progerin Detection in Untreated Patients

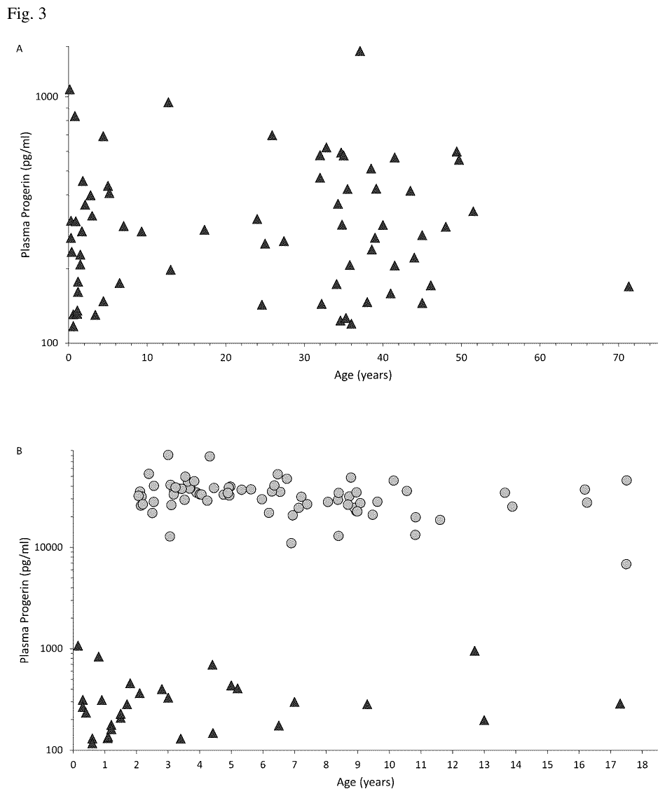

NonHGPS Plasma: A set of nonHGPS plasma (N=69; 39M, 30F) was assessed (FIG. 3, panel A). This set of samples consisted of 33 donors evaluated for HGPS or nonHGPS progeroid laminopathies (PL) using LMNA±ZMPSTE24 mutational analysis, 32 healthy relatives of those being evaluated for mutations, and 4 plasma samples from healthy controls purchased commercially. Within the donors being evaluated for HGPS or PL, none had progerin-producing mutations. Overall, mean progerin was 351±251 pg/mL (range=ND−1534). Mean donor age was 23.3±18.7 years (range 0.2-71.3). There was no correlation between donor age and progerin (r=0.3, p=0.81). In addition, progerin levels in a separate group of nonHGPS patients with congestive heart failure (N=10; 5M, 5F; progerin 325±157 pg/mL) or kidney disease (N=10; 4M, 6F; progerin 361±227 pg/mL) were not elevated (p>0.05). However, the small number of samples is highly preliminary and demonstrates that progerin is present, but an exponentially larger sample size must be evaluated to fully assess the relationship between progerin and generalized aging and diseases of aging such as cardiovascular and kidney disease. Thus the hypothesis that circulating progerin is related to generalized aging and diseases of aging such as cardiovascular and kidney disease remains viable, given that Progeria is a premature aging disease and is a model that serves as an exaggerated early signpost for relevant features of aging and diseases of aging.

Plasma Progerin in Drug-naïve Patients with HGPS: We assessed the profile of plasma progerin in children with HGPS at baseline, prior to trial drug initiation (FIG. 3, panel B). Mean progerin was 33,249±12,299 pg/mL overall (N=74); with females 33,237±11,797 (N=37) and males 33,285±13,035 (N=37). There was no association between progerin and sex. Average HGPS progerin was 95-fold increased over nonHGPS (p<0.0001). For individual trials, mean baseline treatment-naïve progerin was 27,572±7,542 pg/mL for ProLon1 (N=26), 31,464±16,958 pg/mL for Triple Therapy (N=13) and 38,154±11,546 pg/mL for ProLon2 (N=35). Though there were no significant differences in progerin when comparing ProLon1 or ProLon2 with Triple Therapy (p=0.44 and 0.12, respectively), baseline values were higher in ProLon2 vs. ProLon1 (p<0.0001). A subgroup comparison consisting of all age- and sex-matched controls also demonstrated highly significant difference between HGPS (progerin=34,512±14,432 pg/mL; N=25) and nonHGPS control levels (progerin=377±226 pg/mL; N=13; p<0.0001).

Plasma Progerin versus Age: While most patients donated a single baseline sample just prior to clinical trial drug administration at the BCH trial site, a subset of patients also donated a blood sample as part of a natural history study conducted prior to the BCH trial donation, at the National Institutes of Health (NIH) Clinical Center (Merideth et al.); thus allowing longitudinal progerin assessment free from the influence of drug therapy. These samples were subsequently submitted to the PRF Cell and Tissue Bank. NIH study blood samples were processed similarly to the clinical trial samples. The average time between blood draws was 1.6±0.5 years (range 0.9-2.3). Average initial and follow-up plasma progerin (N=13) was 29,221±7,772 vs 33,272±11,959 pg/mL, respectively; there was no significant difference in progerin with time (p=0.14).

### Example 3: Plasma Progerin Assay for Determining HGPS Treatment Efficacy

This example examines the possibility of using the developed progerin immunoassay to determine efficacy of treatment of HGPS, such as with the FTI lonafarnib.

Exogenous Lonafarnib Does Not Interfere with the Progerin Assay: To assess whether changes in plasma progerin would truly reflect the influence of lonafarnib therapy on plasma progerin levels, we assessed whether lonafarnib itself interferes with progerin assay readings. Because the Cmax determined from a pharmacokinetics study in children with HGPS treated with lonafarnib during a clinical trial was 2.64 μg/mL (Gordon, 2012), plasma samples were spiked with 7.92 μg/mL lonafarnib, which is 3×Cmax. Mean progerin was unaffected by spiking with lonafarnib in DMSO as compared with the control conditions (p=0.198; Table 5).

TABLE 5
Test of Exogenous Lonafarnib Interference with Plasma Progerin Detection

| | Rep 1 | Rep 2 | Rep 3 | Mean | | | |
|---|---|---|---|---|---|---|---|
| | progerin | progerin | progerin | progerin | | | Testing |
| Plasma ID | pg/mL | pg/mL | pg/mL | pg/mL | SD | % CV | condition |
| HGPl204 | 25,841 | 25,702 | 27,676 | 26,407 | 1,102 | 4 | DMSO |
| HGPl204 | 26,681 | 25,854 | 26,406 | 26,314 | 421 | 2 | lonafarnib |
| | | | | | | | in DMSO |
| HGPl201 | 48,868 | 46,242 | 51,008 | 48,706 | 2,387 | 5 | DMSO |
| HGPl201 | 47,302 | 48,300 | 43,628 | 46,410 | 2,461 | 5 | lonafarnib |
| | | | | | | | in DMSO |
| HGPl194 | 25,218 | 25,463 | 30,136 | 26,939 | 2,771 | 10 | DMSO |
| HGPl194 | 22,562 | 24,115 | 22,378 | 23,018 | 954 | 4 | lonafarnib |
| | | | | | | | in DMSO |

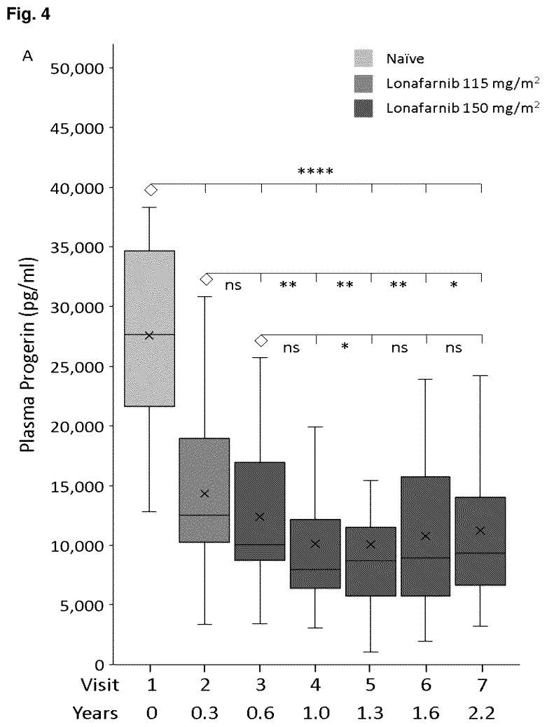

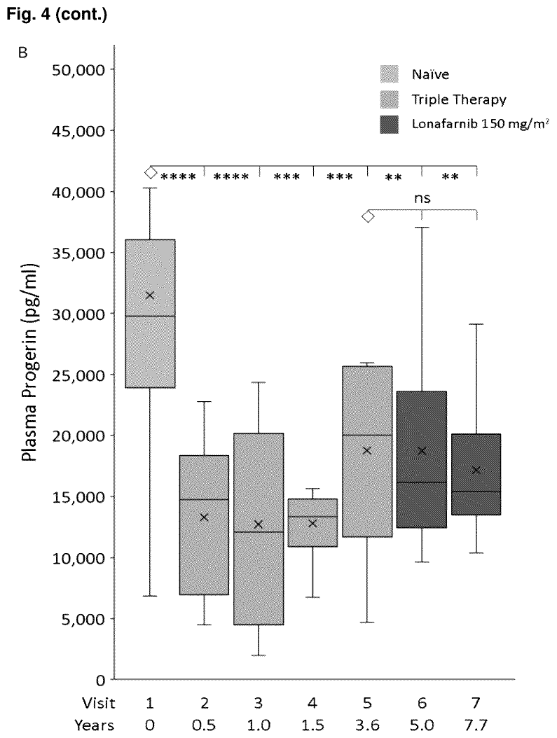

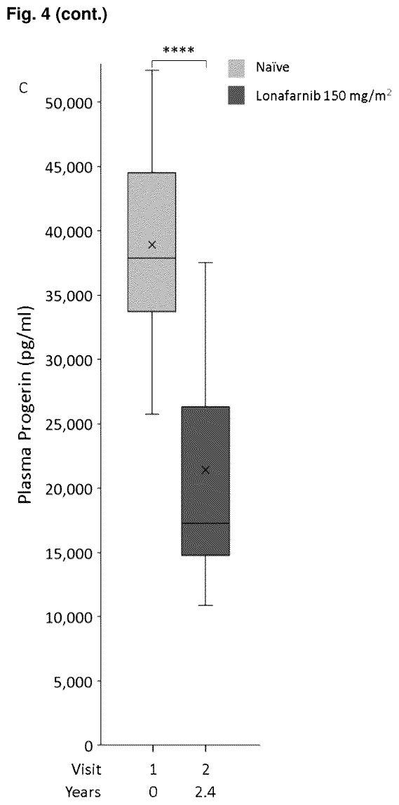

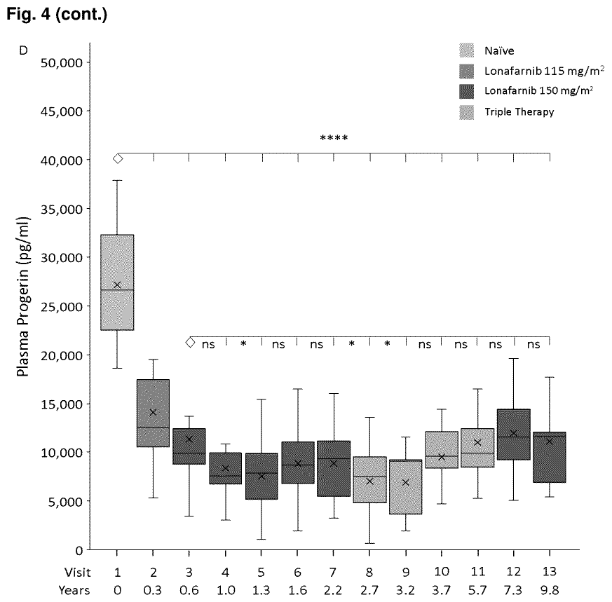

Changes in Progerin with Lonafarnib Therapy: ProLon1 was the first clinical trial administering lonafarnib to children with HGPS and featured several differences from Triple Therapy and ProLon2. Patients visited the trial site and had blood collections every 4 months for a total of 2.2±0.1 years (7 visits), compared with every 6 months for a total of 3.5±0.2 years during Triple Therapy (5 visits) and baseline plus end-of-study at 2.4±0.6 years on therapy for ProLon2 (2 visits). In addition, patients in ProLon1 initiated therapy at 115 mg/m² for 4 months before elevating to 150 mg/m². Both Triple trial and ProLon2 initiated patients at 150 mg/m². Sample number varied across the individual studies and visits; comparisons between study visits included matched plasma sets only. Progerin changes within and between separate but sequential trials were compared (FIG. 4, panels A-D). Overall, average on-therapy plasma progerin decreased from baseline untreated by 38%, from 32,726±12,659 to 20,211±10,190 pg/mL (p<0.0001).

ProLon1 Progerin Changes with Lonafarnib Therapy: Average progerin decreased from baseline by 48% at month 4, during the dosing period using 115 mg/m² lonafarnib (N=25, p<0.0001, FIG. 4, panel A). For each of the 5 remaining patient visits where lonafarnib dose was 150 mg/m², average decreases from baseline ranged between 50-62% (N=22-25 patients, all p<0.0001). Importantly, at the 8-month trial visit, 4 months after transitioning from 115 to 150 mg/m² dosing, progerin was further decreased to levels close to significance (N=25, p=0.06), and at each of the 4 subsequent trial visits, progerin was significantly decreased while taking 150 mg/m² of lonafarnib over the 115 mg/m² dose (N=22-25 patients, all p<0.05). There were no significant differences between average progerin at the 8-month 150 mg/m² visit and subsequent visits at the same drug dose (N=22-25 patients, all p≥0.05). This dose response demonstrates the assay's ability to detect an incremental but significant benefit of 150 mg/m² dosing over 115 mg/m².

Progerin Changes with Lonafarnib+Pravastatin+Zoledronic acid Therapy (Triple Therapy): At the first on-therapy trial visit after 6 months, average progerin decreased from baseline by 41% (N=13, p=0.0018; FIG. 4, panel B). Thereafter, average progerin remained significantly decreased from baseline by 35-47% (N=12-13 patients, visits 3-5, p=0.0015-0.0058).

After the formal Triple Therapy period ended, pravastatin and zoledronic acid were discontinued and 10 patients completed a lonafarnib monotherapy extension for an additional 4.1±0.5 years (range 3.41-4.65; FIG. 4, panel B). For these 10 patients, there were no significant differences in average progerin between consecutive Triple Therapy and on-treatment lonafarnib monotherapy extension trial visits (p=0.99).

ProLon2 Progerin Changes with Lonafarnib Therapy: Average progerin decreased from baseline by 36.7% during the dosing period (N=26, p<0.0001; FIG. 4, panel C).

Long-term Lonafarnib Therapy Effect on Progerin: A subgroup of 13 subjects were treated continuously with lonafarnib, without treatment breaks, as part of ProLon1, Triple Therapy Trial and lonafarnib monotherapy extension of Triple Trial (FIG. 4, panel D). This constitutes an average of 9.8±0.5 (range 9.0-10.3) years of continuous lonafarnib therapy. Average progerin decreased from baseline by 48% at month 4 (p<0.0001), during the dosing period using 115 mg/m². For all subsequent time points, average decreases from baseline during 150 mg/m² dosing ranged from 56-74% (p<0.0001). Compared to the first time point on 150 mg/m² (time point #3 at 0.66 yr), time points 5, 8 and 9 were significantly lower (p<0.05), and all other time points (4, 6, 7, 10-13) were similar to time point #3 (p≥0.05).

Diurnal Variation in Progerin using Pharmacokinetics Studies: Plasma was collected at trough, and post-dose hours 1,2,4,6,8 for pK studies during ProLon1 at lonafarnib doses 115 mg/m² and 150 mg/m² (Gordon, 2012), as well as during Triple Therapy (Gordon, 2016). Within each of these sample sets, progerin did not demonstrate significant changes in post-dose vs. trough levels for ProLon1 at 150 mg/m² and for Triple Therapy, indicating that progerin does not demonstrate diurnal variation (all p≥0.05). For ProLon1 lonafarnib dose of 115 mg/m², average progerin trough was similar to 1-hour post-dose (p=0.2647), but was significantly lower than 2-, 4- and 8-hours post-dose and differed on average by about 3,000 pg/mL (all p<0.05).

### Example 4: Progerin Level Is Strongly Associated With Survival

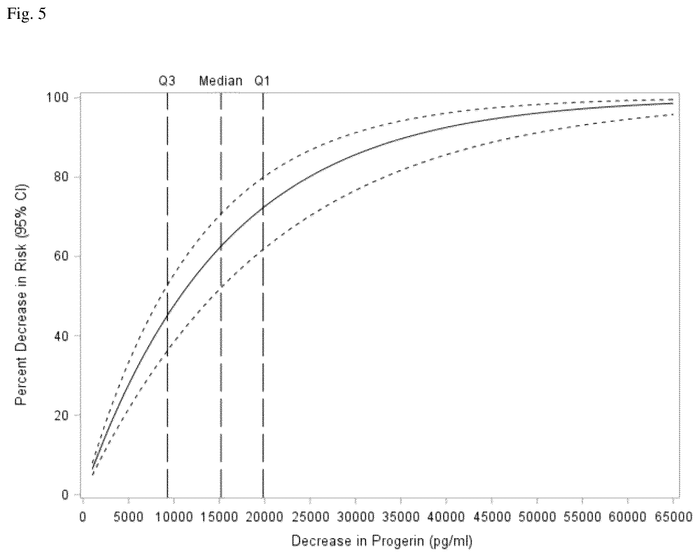

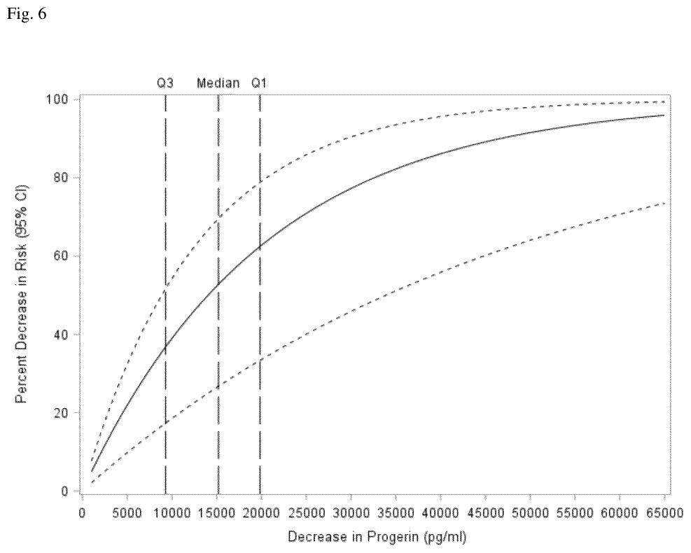

This example assesses the suitability for plasma progerin to serve as a predictive biomarker for survival. First, change in mortality risk per change in measured progerin values was estimated directly from the joint model relating these two measures. All patients with plasma samples were included regardless of whether therapy was implemented, length of therapy, or follow-up time (N=74; 26 deceased and 47 living as of Apr. 1, 2022). Progerin levels were significantly related to risk of death (p<0.0001; FIG. 5). For example, a 1,000 pg/mL decrease in plasma progerin corresponded to a 6.3% decrease in the risk of death (95% CI of 4.7-7.8%), whereas a 10,000 pg/mL decrease in plasma progerin, corresponded to a 47.6% (95% CI: 38.4-55.4%) decrease in the risk of death. Progerin was modeled as a function of length of therapy, length of therapy squared, sex, and age at baseline. The joint model adjusted for length of therapy. Additional analysis using standard time-dependent Cox modeling, which does not consider potential measurement error in the longitudinal covariate (progerin), as well as subgroup analysis using only those patients with at least one on-therapy plasma sample, yielded similar results (FIG. 6, Table 6).

TABLE 6
Estimated Effect of Change in Plasma Progerin
on Patient Risk of Mortality in Classic HGPS*

| | | Decreased (CI) | | |
|---|---|---|---|---|
| | | Risk of Mortality | | |
| | | 1,000 | 10,000 | |
| | | pg/mL | pg/mL | |
| Minimum Patient | Statistical | Progerin | Progerin | P |
| Inclusion Criteria | Analysis | Decrease | Decrease | value |
| Naïve progerin | Cox Time- | 4.8% | 39.0% | 0.0008 |
| (N = 74) | Dependent | (2.0, 7.5) | (18.5, 54.3) | |
| (9 patients with | | | | |
| only naïve levels; | Joint | 6.3% | 47.6% | <0.0001 |
| 65 patients | model | (4.7, 7.8) | (38.4, 55.4) | |
| with naïve | | | | |
| baseline plus | | | | |
| on-therapy levels) | | | | |
| Naïve baseline | Cox Time- | 4.1% | 34.2% | 0.0370 |
| and on-therapy | Dependent | (0.3, 7.8) | (2.5, 55.6) | |
| progerin (N = 65) | | | | |
| | Joint | 5.5% | 43.0% | 0.0008 |
| | model | (2.3, 8.5) | (20.9, 58.9) | |

*Effect of change in progerin on survival was calculated using the hazard ratios estimated from the Cox and joint models, relating measured plasma progerin to mortality.

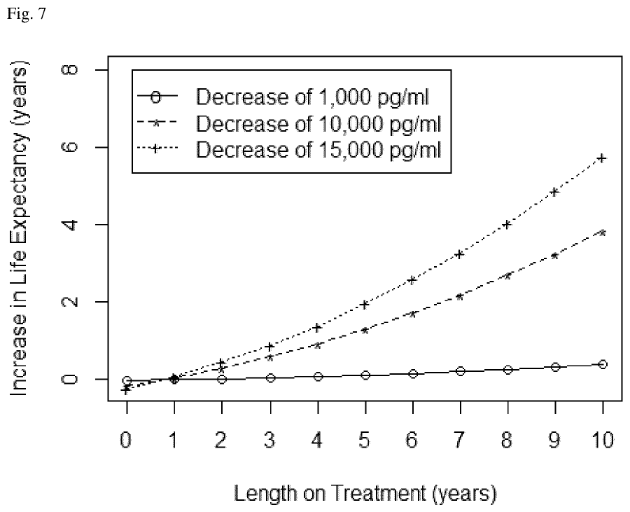

Next, the relationship between lonafarnib treatment duration and change in life expectancy with extent of change in plasma progerin was assessed (FIG. 7, Table 7). This analysis included samples from patients with a baseline pre-therapy and at least one on-treatment progerin measurement (N=65 patients). Life expectancy incrementally improved with both extent of plasma progerin decrease, and time spent at lower progerin level. Three representative wide-ranging examples of on-therapy progerin decreases (1,000, 10,000 and 15,000 pg/mL) spanning 10 years on treatment are graphed in FIG. 7. Patient lifespan increased in all 3 examples.

TABLE 7
Lifespan Expectancy Is Positively Associated with Duration
of Time at Therapy-induced Lower Plasma progerin

| Time at | Change in Lifespan Expectancy | | |
|---|---|---|---|
| Designated | with Decrease in progerin (years) | | |
| Lower Progerin | Per 1,000 | Per 10,000 | Per 15,000 |
| Level (years) | pg/mL | pg/mL | pg/mL |
| 1 | 0.00546 | 0.0546 | 0.0819 |
| 2 | 0.03024 | 0.3024 | 0.4536 |
| 3 | 0.05934 | 0.5934 | 0.8901 |
| 4 | 0.09276 | 0.9276 | 1.3914 |
| 5 | 0.1305 | 1.305 | 1.9575 |
| 6 | 0.17256 | 1.7256 | 2.5884 |
| 7 | 0.21894 | 2.1894 | 3.2841 |
| 8 | 0.26964 | 2.6964 | 4.0446 |
| 9 | 0.32466 | 3.2466 | 4.8699 |
| 10 | 0.384 | 3.84 | 5.76 |

### Example 5: Discussion

The development of clinically relevant biologic markers of disease is critical to identifying and implementing effective treatments for children and young adults with HGPS. As described herein, validation of the progerin biomarker that directly quantifies the disease-causing protein has the potential to significantly improve both preclinical and clinical trial assessments of new treatment approaches for this universally fatal disease. Moreover, plasma progerin's quantitative relationship to patient survival solidifies its clinical relevance.

Cultured fibroblasts, urine and serum underwent preliminary assay testing to assess viability of future assay development for additional tissue types. Progerin was detected in all these sample types isolated from HGPS patients.

HGPS fibroblast lysate (assayed in triplicate) yielded progerin level of 14.0±0.3 pg progerin/μg total protein. Progerin was below LLoQ in control fibroblast cell lysate (assayed in triplicate). Urine sediment and supernatant were assessed from nonHGPS controls (N=2), and HGPS patients (N=2). In nonHGPS controls (N=2), progerin was below LLoQ in sediment and supernatant. In each of two HGPS samples, sediment contained 30,986 and 13,996 pg progerin/mg total protein, and supernatant contained 10,591 and 8,009 pg progerin/mg total protein, respectively. When normalizing to urinary creatinine, supernatants contained 1,546.5 and 447.1 pg progerin/mg creatinine, respectively. Finally, serum and plasma were isolated from the same blood draw (N=15, 7 Male, 8 Female, all classic HGPS). Average detected [progerin] was similar (p=0.26), with mean serum [progerin] 25,963±11,075 pg/ml and mean plasma [progerin] 28,697±9,313 pg/ml.

Progerin has not previously been detected in plasma, serum, urine, or cell lysates, all of which can be sampled longitudinally. Previous investigations of treated and untreated HGPS patient serum and plasma have therefore relied on identifying novel potential biomarkers that lie downstream of the disease-causing protein, progerin and reflect either the direct or indirect effects of this toxic protein. We have described herein a sensitive, reproducible immunoassay for detecting progerin was developed and subsequently used to explore progerin's presence in plasma, filling a major gap in the field of progeria research. Importantly, the assay did not detect lamin A, progerin's normal protein counterpart. The sensitivity and low-volume format of the assay allowed analysis of individual HGPS patient samples without the need for pooling, which is important for this population of small and fragile children where blood draw volume must be minimized for safety.

High assay sensitivity also permits progerin quantification in nonHGPS individuals, which will facilitate future research on aging and aging-related diseases such as cardiovascular diseases, insulin resistance (pre-diabetes), alopecia and lipodystrophy. Progerin levels in plasma from untreated HGPS patients were on average 95-fold higher than in nonHGPS plasma, though nonHGPS values were sometimes below the assay's sensitivity (<LLoQ). This agrees with a prior autopsy study that detected similar ratios of progerin in the vascular wall using immunofluorescence. Thus, this assay greatly expands the possibilities for preclinical and clinical research into HGPS and generalized aging.

Though plasma was chosen for primary assay development, other noninvasive biological samples such as urine or saliva from patients and animal models, as well as cell culture material and disease tissue, can be evaluable using a similar approach as described herein. A comprehensive study of progerin's relative presence between different organs and tissue types using the assay developed here on either human autopsy specimens or animal models of HGPS would be highly useful to understand how plasma progerin reflects relative tissue contributions to disease, both HGPS and progerin-related aging pathologies.

The limited dataset of samples collected longitudinally from patients naïve-to-therapy indicates progerin deposition into the plasma remains steady over the 2-year period tested. In addition, on average the plasma levels in the treated patient group were decreased by almost 40% from baseline and stayed lower for up to 10 years. The pathophysiology behind these longitudinal findings deserves further investigation. There is evidence from human autopsy tissue that progerin can accumulate in tissues with time, implying that progerin production per cell might increase with aging. However, aging in both humans and HGPS mice also results in a relatively acellular vascular media where proteoglycan replaces healthy tissue and vascular stiffening ensues. Other progerin-producing tissues such as liver and kidney do not seem as susceptible to cell loss. A better understanding of the balance between cellular progerin production, cell loss, and the relative contributions of various organs to plasma progerin is needed for insight into the interplay between plasma progerin and tissue pathology, and for a more comprehensive assessment of drug activity.

The only approved drug for HGPS and select progeroid laminopathies is the farnesyltransferase inhibitor lonafarnib (Zokinvy), placing Progeria among the less than 5% of rare diseases with an approved medication. Its clinical trial success was defined by outcomes measured after at least 2 years of treatment, presumably when the sensitivity of clinical testing was adequate to detect change. Importantly, the presence of lonafarnib did not interfere with progerin detection. Lonafarnib therapy significantly decreased plasma progerin levels within 4 months, portending a potential shorter-term readout of drug efficacy in future trials with other drugs, depending on the mechanism of drug effect. This would decrease the risk of being exposed to interventions that may not be effective, such as in the Triple Therapy Trial where adding pravastatin and zoledronate did not enhance lonafarnib's beneficial effects on disease. Moreover, earlier identification of an effective therapy could be accelerated with regulatory use of progerin as a surrogate endpoint, in combination with a longer-term clinical outcome measure. Additionally, a steady state on-therapy reduction in progerin for up to 10 years implies that there was no measurable rebound in progerin. Thus, progerin levels in blood are an excellent candidate for both short and long-term treatment trial outcomes.

A biomarker is a defined characteristic that is measured as an indicator of normal biological or pathogenic processes, or biological responses to an exposure or intervention, including therapeutic interventions. This study demonstrates that plasma progerin is an indicator of both pathogenic process and of clinical response to treatment. Given the previously theorized association between progerin and aging in non-HGPS subject, the described bioassay is applicable in the context of general aging and related conditions as well.

When untreated HGPS patients were included in the survival analysis, plasma progerin levels were associated with mortality risk regardless of whether a treatment had been implemented. Further, plasma progerin not only decreased in response to treatment with lonafarnib, but that decrease was strongly associated with and incrementally conferred survival benefit. For example, since on average trial patients' plasma progerin decreased by more than 14,000 pg/mL, life expectancy for the 13 patients on therapy for over 10 years is estimated to increase by almost 6 years. This represents a greater than 40% increase in average lifespan, from 14.5 to over 20 years of age. Thus, the critical statistical and biological evidence for use of progerin as a biomarker reasonably likely to predict clinical benefit is present.

There are a variety of treatment strategies in development for HGPS that may benefit greatly from an assay capable of detecting progerin. These potential treatments are targeted to alter progerin levels using widely varying mechanisms of action, including genetic editing, RNA-based therapies, and small molecules that target progerin protein's post-translational processing, interactions with lamin A, and/or rate of autophagy.

When evaluating the extent to which plasma progerin change reflects disease improvement, the mechanism of drug action must be considered. For example, genetic editing permanently corrects a cell's mutation, so that its progeny will also be normal. In a HGPS murine study using this approach, a 140% increase in lifespan was achieved with only 20-60% mutation correction of cells in tested tissues. Presumably part of the efficacy of this correction was engendered by a healthy subpopulation of cells and their creation of a healthier microenvironment, which had ramifications for adjacent cells that were not corrected. Lonafarnib, which caused on average a 38% decrease in plasma progerin, penetrates all cell types and would therefore inherently decrease progerin on a large scale, even though the cells it affects are still diseased and continue to produce new progerin molecules. In this context, the gene editor might have less of an effect on plasma progerin than lonafarnib, while still producing greater clinical benefit. Thus, the hypothesis would be that any decrease in plasma progerin reflects a benefit to disease, but the relative magnitude of the decrease between different types of therapies may not be a valid way to assess the extent of benefit.

There are several study limitations. First, plasma is a “sink” for deposition of progerin from multiple organs and does not differentiate the relative contribution from organs of major disease interest such as the heart and vasculature. However, there is evidence that drug delivery to organs other than vasculature can be beneficial, as extravascular delivery of gene therapy improved both healthspan and increased lifespan in HGPS mice. Thus, the natural history of HGPS is not understood well enough to eliminate non-cardiovascular organs as pivotal contributors to phenotype.

Treatment with lonafarnib improves some disease features and significantly extends lifespan in progeria, but affected individuals still die of accelerated atherosclerosis and heart failure in their teens and twenties. There is a critical need for improved treatments and a cure. The progerin assay established in this study will enable early identification of new treatments that effectively target progerin, either directly or indirectly.

### REFERENCES

Bar D Z, et al., J Med Genet. 2017; 54:212-216.
Benedicto I et al., Cells. 2021; 10.
Cabral W A et al., Aging Cell. 2021:e13457.
Camafeita E et al., Int. J. of Molec. Sci. 2022, 3, 11733.
Dafni U., Circ Cardiovasc Qual Outcomes. 2011; 4:363-371.
De Sandre-Giovannoli A et al., Progress in molecular and subcellular biology. 2006; 44:199-232.
Erdos et al., Nature Medicine. 2021. 27, 536-545.
Eriksson M et al., Nature. 2003; 423:293-297.
Fong L G et al., The Journal of Clinical Investigation. 2006; 116:743-752.
Gordon L B et al., PNAS. 2012; 109:16666-16671.
Gordon L B et al., Circulation. 2014; 130:27-34.
Gordon L B et al., Circulation. 2016; 134:114-125.
Gordon L B et al., JAMA. 2018; 319:1687-1695.
Gordon L B et al., Circulation. 2023; 147:1734-1744.
Hisama F M et al., American Journal of Medical Genetics Part A. 2011; 155A:3002-3006.
Kang et al., Nature Comm. Biol. 4, Article 5, 2021.
Koblan et al., Nature. 2021. 589, 608-614.
Macicior et al. International journal of molecular sciences. 2021; 22.
Merideth M A et al., NEJM. 2008; 358:592-604.
Murtada S I et al., J R Soc Interface. 2020; 17:20200066.
Puttaraju et al., Nature Medicine. 2021. 27, 526-535.
Scaffidi P et al., Nature Medicine. 2005; 11:440-445.
Todd J et al., Clin Chem. 2007; 53:1990-1995.
Yang Z et al., Comput Methods Programs Biomed. 2021; 207:106155.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for prognosis and/or detection of a progerin-related aging pathology in a subject, comprising:
providing a sample from a subject;
quantitating the concentration of progerin in in the sample with a quantitative immunoassay, wherein the quantitative immunoassay detects progerin but does not detect wildtype lamin A protein; and
comparing the concentration of progerin in the sample with an progerin-related aging pathology-positive control,
wherein the concentration of progerin in the sample that is at or above the concentration of progerin in the positive control diagnoses the pathology in the subject, or indicates a negative prognosis; and
wherein the concentration of progerin in the sample that is below the concentration of progerin in the positive control indicates a positive prognosis.

2. The method of claim 1, wherein the sample is a liquid sample selected from plasma, serum, urine, cell extract, and saliva.

3. The method of claim 1, wherein the quantitative immunoassay comprises:
contacting the sample with a capture antibody that binds to both wildtype lamin A and progerin, resulting in a mixture of captured lamin A and captured progerin;
separating the mixture of captured lamin A and captured progerin from the sample;
contacting the mixture of captured lamin A and captured progerin with a progerin-specific detection antibody comprising a detectable label, thereby binding the captured progerin but not the captured lamin A with the detection antibody;
separating the detectable label from the mixture with captured lamin A; and
detecting the detectable label, and thereby quantitating the amount of progerin in the sample.

4. The method of claim 1, wherein the control is from the same subject at an earlier time point.

5. The method of claim 1, wherein the lower limit of quantitation of the quantitative immunoassay is 59 pg/ml.

6. The method of claim 1, wherein the upper limit of quantitation of the quantitative immunoassay is 30,000 pg/ml.

7. The method of claim 1, further comprising repeating the method one or more times after the subject undergoes a treatment for the pathology, wherein a decrease in progerin concentration following the treatment indicates a positive prognosis.

8. The method of claim 7, wherein a greater decrease in progerin concentration indicates an greater increase in life expectancy.

9. A method for determining the efficacy of a treatment for a progerin-related aging pathology, comprising:
providing a pre-treatment sample from a subject, that is taken prior to administration of a treatment for a progerin-related aging pathology;
determining the concentration of progerin in the pre-treatment sample with a quantitative immunoassay, wherein the quantitative immunoassay detects progerin but does not detect wildtype lamin A protein;
providing a post-treatment sample from the subject, that is taken during or after administration of a treatment for the pathology;
determining the concentration of progerin in the post-treatment sample with the quantitative immunoassay; and
comparing the pre-treatment concentration of progerin with the post-treatment concentration of progerin, wherein a significant decrease in progerin concentration in the post-treatment sample indicates that the treatment is effective.

10. The method of claim 9, wherein the pre-treatment sample and the post treatment sample are a liquid sample selected from plasma, serum, cell extract, urine, and saliva.

11. The method of claim 10, further comprising providing one or more additional post-treatment samples that are taken from the subject at time points subsequent to the initial post-treatment sample, and wherein a continued or further decrease in progerin concentration indicates continued efficacy of the treatment.

12. The method of claim 10, wherein the quantitative immunoassay comprises:
contacting the sample with a capture antibody that binds to both wildtype lamin A and progerin, resulting in a mixture of captured lamin A and captured progerin;
separating the mixture of captured lamin A and captured progerin from the sample;
contacting the mixture of captured lamin A and captured progerin with a progerin-specific detection antibody comprising a detectable label, thereby binding captured progerin but not captured lamin A with the detection antibody;
separating the detectable label from the mixture with captured lamin A;
detecting the detectable label, and thereby quantitating the amount of progerin in the sample.

13. The method of claim 10, wherein the lower limit of quantitation of the quantitative immunoassay is 59 pg/ml.

14. The method of claim 10, wherein the upper limit of quantitation of the quantitative immunoassay is 30,000 pg/ml.

15. The method of claim 10, wherein the treatment comprises a farnesyl transferase inhibitor.

16. A method for treatment of a progerin-related aging pathology in a subject, comprising:
providing a sample from a subject;
quantitating the concentration of progerin in in the sample with a quantitative immunoassay, wherein the quantitative immunoassay detects progerin but does not detect wildtype lamin A protein;
comparing the concentration of progerin in the sample with a progerin-related aging pathology-positive control, wherein if the concentration of progerin in the sample indicates that the subject has the pathology; and
if the subject has the pathology, administering to the subject a treatment for the pathology that lowers progerin concentration.

17. The method of claim 16, wherein the lower limit of quantitation of the quantitative immunoassay is 59 pg/ml.

18. The method of claim 16, wherein the upper limit of quantitation of the quantitative immunoassay is 30,000 pg/ml.

19. The method of claim 16, wherein the treatment for the pathology comprises administering a farnesyl transferase inhibitor to the subject.

20. The method of claim 19, wherein the farnesyl transferase inhibitor is lonafarnib.
